(19) United States
(12) Patent Application Publication
(10) Pub. No.: US 2009/0214486 A1
(43) Pub. Date: Aug. 27, 2009

(54) Serum Response Factor and Myocardin Control Alzheimer Cerebral Amyloid Angiopathy

(76) Inventors: Berislav V. Zlokovic, Rochester, NY (US); Joseph M. Miano, Rochester, NY (US)

Correspondence Address: NIXON & VANDERHYE, PC, 901 NORTH GLEBE ROAD, 11TH FLOOR, ARLINGTON, VA, 22203, US

(21) Appl. No.: 12/084,774

(22) PCT Filed: Nov. 14, 2006
(86) PCT No.: PCT/US06/44143

§ 371 Date: Feb. 5, 2009

Related U.S. Application Data
(60) Provisional application No. 60/735,965, filed on Nov. 14, 2005.

(51) Int. Cl. A61K 35/12; A61K 31/7105; C12Q 1/68; C12Q 1/02; C07H 21/02

(52) U.S. Cl. 424/93.7; 514/44 A; 435/6; 435/29; 536/24.5

(57) ABSTRACT

Cerebral amyloid angiopathy is involved in Alzheimer dementia through reduction in arterial blood flow that may impair protein synthesis, which is required for learning and memory, and lower the threshold for ischemic injury. Elevated serum response factor (SRF) or myocardin (MYOCD) activity in subjects afflicted by or at risk for development of Alzheimer's disease (AD) promotes a “vascular smooth muscle cell” (VSMC) hypercontractile phenotype in brain arteries and enhance accumulation of Aβ in the vessel wall. This, in turn, can initiate a disease process in cerebral arteries which can cause brain arterial hypoperfusion and neurovascular uncoupling, that are commonly seen in AD. Thus, SRF and MYOCD represent novel targets for treating arterial dysfunction associated with cognitive decline in AD.

### CROSS-REFERENCE TO RELATED APPLICATIONS

This application claims the benefit of provisional U.S. Application No. 60/735,965, filed Nov. 14, 2005.

### FEDERALLY-SPONSORED RESEARCH OR DEVELOPMENT

The U.S. Government has certain rights in this invention as provided for by the terms of NIH grant AG023084 or AG023993 from the Department of Health and Human Services.

### BACKGROUND OF THE INVENTION

This invention relates to Alzheimer's disease (AD) and its pathogenesis by addressing its etiology, and thereby ameliorating or reversing its hypercontractile phenotype. Products and processes used therein are provided.

Alzheimer dementia is characterized by the progressive cognitive decline associated with neurovascular dysfunction^{1,2}, impaired brain clearance of Aβ toxin^{20,22,23}, and neuronal injury and loss^{19,20}. Arterial hypoperfusion may precede Aβ accumulation and cerebral atrophy in animal models of AD²⁴⁻²⁶ and in AD patients²⁷⁻³⁰. Cerebral arteriopathy reduces blood flow to the brain. It is associated with cognitive decline and Aβ accumulation in the vessel wall, which is known as cerebral amyloid angiopathy (CAA)^{3,4}.

In AD, we show cerebral vascular smooth muscle cells (VSMC), which regulate arterial flow to the brain by controlling the diameter of pial and intracerebral arteries^{1,2}, express in vitro and in vivo high levels of serum response factor (SRF) and myocardin (MYOCD), two interacting transcription factors which orchestrate a SMC phenotype^{13,14}. AD VSMC overexpress the SRF-MYOCD-regulated contractile proteins^{13,18} and exhibit hypercontractility. MYOCD gene transfer to human cerebral VSMC induces an AD-like hypercontractile arterial phenotype, whereas silencing the SRF gene in AD VSMC normalizes contractile protein content and cell contractility. Transduction of mouse arteries with MYOCD gene diminishes endothelial-dependent arterial vasodilation and enhances arterial response to vasoconstrictors. Exposure to Alzheimer toxin, amyloid β-peptide (Aβ)^{19,20}, in vitro or in an Aβ overproducing mouse model of AD²¹, did not affect SRF expression in cerebral VSMC, whereas silencing the SRF gene in AD VSMC improved clearance of Aβ aggregates consistent with upregulation of the Aβ lipoprotein clearance receptor^{22,23}. Thus, SRF-MYOCD gene activation in cerebral VSMC may initiate Alzheimer arteriopathy associated with cognitive decline.

Therefore, it is an objective of the invention to provide a treatment for a subject who is affected by Alzheimer's disease (therapy) or who is at risk for its development (prophylaxis). A long-felt need is addressed thereby to reduce the number and/or severity of symptoms associated with Alzheimer's disease. Further objectives and advantages of the invention are described below.

### SUMMARY OF THE INVENTION

An objective is to address (e.g., reverse) a hypercontractile phenotype associated with Alzheimer's disease by reducing serum response factor (SFR) and/or myocardin (MYOCD) regulated gene expression in at least a cell of a subject's vasculature. The reduction in SRF/MYOCD-regulated gene expression may be by achieved by technologies such as, for example, antisense inhibition, RNA interference, trans-dominant interference, and other inhibitors of gene activation or regulation in the SRF-MYOCD transcriptional pathway. Such treatment may also cause decreased expression of one or more contractile proteins in the cell and/or increased blood flow in the vasculature. Treatment of a subject may be performed one or more times in vivo or ex vivo with a transplantable cell(s) from an autologous or heterologous (i.e., allogenic or xenogenic) source.

Another objective is to diagnose Alzheimer's disease. A sample of body fluid or tissue from a subject is analyzed for SRF and/or MYOCD expression at the level of transcription, translation, or protein activity. Increased expression is a risk factor for the existence or development of Alzheimer's disease. Additional risk factors may be vascular hypercontractility, amyloid angiopathy, reduced blood flow, and any combination thereof. The body fluid may be brain interstitial fluid (ISF) or cerebrospinal fluid (CSF) containing cells that express SRF or MYOCD, or surrogate sources of endothelial (especially smooth muscle) cells. The tissue may be brain or other central nervous system tissues such as cerebral arteries, leptomenengial vessels, and temporal arteries as well as other endothelial (especially smooth muscle) cells.

The subject of treatment or diagnosis is preferably an animal model of Alzheimer's disease, a human patient afflicted with Alzheimer's disease, or a human patient with one or more risk factors for developing Alzheimer's disease. The cell is preferably a smooth muscle cell.

Further aspects of the invention will be apparent to a person skilled in the art from the following description of specific embodiments and the claims, and generalizations thereto.

### BRIEF DESCRIPTION OF THE DRAWINGS

FIG. 1 shows SRF/MYOCD and contractile protein expression and activity in Alzheimer's disease brain arterial smooth muscle cells. (A) Western blots for smooth muscle myosin heavy chain (SM-MHC), a full length SRF (upper arrow) and its dominant negative isoforms (lower arrows), SM α-actin, SM22α, and SM-calponin in AD and age-matched control VSMC. (B-C) Relative levels of expression of VSMC contractile proteins (B) and SRF isoforms (C) in AD (open bar) and controls (closed bar). (D) QRT-PCR for MYOCD mRNA in VSMC in AD (open bar) and controls (closed bar). (E-F) Cerebral VSMC before (control), during (contraction) and after (relaxation) stimulation with potassium chloride (KCl). (G) Increased contractility of AD VSMC compared to control VSMC determined from 100 cells per culture after stimulation with KCl. Mean±s.e.m. are from 5-8 independent cultures.

FIG. 2 shows SRF/MYOCD and contractile protein expression in Alzheimer's disease brain arterial vessels in situ. (A-B) Double staining for SRF and SM α-actin (A) or MYOCD and SM α-actin (B) in AD or age-matched control brains. (C) Calponin staining in brain tissue in AD vs. controls. Bar=50 μm. (D-F) Relative intensity of SRF-positive (D), MYOCD-positive (E), and SM-calponin-positive (F) vascular profiles in AD (open bars) and controls (closed bars). Mean±s.e.m. from 5 brains per group.

FIG. 3 shows that MYOCD and SRF regulate brain arterial smooth muscle cells contractile phenotype in Alzheimer's disease. (A-C) Western blot analysis for SRF, SM α-actin, SM-calponin, and SM-MHC (A); relative levels of contractile VSMC proteins (B); and VSMC contractility after stimulation with potassium chloride (KCl) (C) in MYOCD-transduced control cerebral VSMC (Ad.MYOCD) (closed bar) or Ad.GFP-transduced VSMC (open bar), (D-F) Western blots for SRF and SM-calponin (D), relative levels of their expression (E), and VSMC contractility after KCl stimulation (F) in Alzheimers disease VSMC transduced with Ad.shSRF (closed bar) or Ad.shGFP (open bar). Mean±s.e.m. from 3-5 independent cultures.

FIG. 4 shows that MYOCD gene transfer in mouse arteries influences their response to vasoactive mediators. (A-B) Cumulative dose-response curves for acetylcholine (A) and phenylephrine (B) in mouse thoracic aortic rings transduced with Ad.MYOCD (solid circle) or Ad.GFP (open circle); *p<0.05. (C) Western blot analysis of smooth muscle myosin heavy chain (SM-MHC) in Ad.MYOCD or Ad.GFP transduced vessels. (Inset) Ex vivo adenoviral-mediated β-galactosidase gene expression in mouse aorta smooth muscle cells layer (left). Scale, 100 μm. Data are mean±s.e.m. from 3-5 mice (*P<0.06).

FIG. 5 shows that SRF gene silencing improves Aβ clearance by Alzheimer's disease brain arterial smooth muscle cells. (A-E) Fluorescence microscopy of multi-spot glass slides coated with Cy3-labeled Aβ42 without cells (A), with control-cerebral VSMC (B), with AD-cerebral VSMC (C), and AD VSMC transduced with Ad.shGFP (D) or Ad.shSRF (E). Cy3-Aβ42 signal and Hoechst-stained nuclei. (F) Relative Cy3-Aβ42 fluorescence intensity in control VSMC with or without receptor-associated protein (RAP) and in AD VSMC alone and transduced with Ad.shGFP or Ad.shSRF. The signal intensity in non-treated, cell-free slides is arbitrarily taken as 100%. (G) LRP levels in control-cerebral VSMC and AD-cerebral VSMC, and in AD-cerebral VSMC transduced with Ad.shGFP and Ad.shSRF. Mean±s.e.m., n=9 measurements from 3 independent cultures per group.

FIG. 6 shows that Ca²⁺ ions are required for cerebral VSMC contraction and that Ca²⁺ fluxes are not altered in Alzheimer's disease VSMC. (A) Relaxation of cerebral VSMC in Ca²⁺-free Krebs solution. (B) Lack of potassium chloride (KCl)-induced contraction in cerebral VSMC cultured in Ca²⁺-free medium. Mean±s.e.m., n=50 cells per culture from 3 different cultures. (C) Ca²⁺ influx in AD and age-matched control cerebral VSMC in response to KCl. Mean±s.e.m. from 3 independent cultures per group.

FIG. 7 shows that Aβ does not affect SRF expression in human cerebral VSMC. (A) Human VSMC were incubated with either normal culture medium or 20 μM Aβ42 oliogomers or aggregates for 8, 24 or 72 hours. SRF levels were determined by Western blot analysis. (B) Relative SRF levels determined by scanning densitometry of the signal intensity of SRF vs. β-actin bands. Mean±s.e.m. from 3 independent cultures per group.

FIG. 8 shows that SRF expression in arterial cerebral microvessels in 18- to 22-month old APPsw^{+/-} mice does not depend on Aβ deposition around blood vessels. (A) SRF-positive vessels (arrows) are only occasionally positive for Aβ (arrowheads) whereas (B) Aβ-positive vessels (arrowheads) are typically negative for SRF immunostaining in 18 and 20-month old APPsw^{+/-} mice, respectively. (C) SRF-positive immunostaining in 20-month old control littermate mouse (arrows) and negative staining for Aβ. Bar=100 μm. (D) Relative SRF intensity/mm² in APPsw^{+/-} mice and age-matched littermate control mice at 18 to 22 months of age. The SRF intensity in control mice was arbitrarily set as 1. Mean±s.e.m. from 3 mice per group.

FIG. 9 shows that SRF expression in cerebral vessels in Alzheimer's disease colocalizes with Aβ deposition. Double immunostaining for SRF (A) and Aβ (B) in brain tissue derived from an AD patient. (C) The merged image shows colocalization of SRF staining with Aβ deposition in cerebral vessels. Bar=25 μm. Data are representative of five AD cases. In contrast, there was relatively little staining for either SRF or Aβ in cerebral vessels in age-matched control individuals (not shown).

### DESCRIPTION OF SPECIFIC EMBODIMENTS OF THE INVENTION

Overexpression of the transcriptional regulators serum response factor (SRF) and its cofactor myocardin (MYOCD) causes an Alzheimer's disease-like hypercontractile phenotype. It may be ameliorated by interrupting SRF and/or MYOCD-regulated gene expression in at least vascular cells (e.g., smooth muscle cells), especially of the brain or artery, and more especially of cerebral artery. In vasculature, vasodilation may be diminished and the response to vasoconstrictors may be enhanced. Increased (as compared to a normal or non-pathological condition) activity of Aβ lipoprotein clearance receptor (LRP) and/or clearance of amyloid β-peptide (Aβ) may be obtained thereby. Alternatively, a pathological condition associated with Alzheimer's disease (AD), such as amyloid angiopathy and its resulting decrease in blood flow, may be ameliorated by interrupting SRF and/or MYOCD-regulated gene expression. Expression of one or more contractile proteins may be decreased or blood flow may be increased in vasculature thereby.

The subject may be a human, other primate, rodent, or other mammal; it may be an animal model of AD, a patient afflicted with AD, or a patient at risk for developing AD. Subjects may be diagnosed by overexpression of at least SRF or MYOCD. For example, a biopsy of endothelial cells may be assayed for SRF or MYOCD mutations, transcriptional activation induced by SRF or MYOCD, expression of SRF- or MYOCD-induced genes, or protein products of SRF or MYOCD. Such diagnostic assay may be performed with an optional determination of amyloid deposits in the biopsy. Material may be obtained from the brain, especially cerebral arteries. Alternative sources for biopsy material are blood or bone marrow cells, leptomenengial vessels, temporal arteries, and other endothelial (especially smooth muscle) cells. Assays may be performed by nucleic acid hybridization or antibody binding techniques: e.g., amplification of transcripts (e.g., RT-PCR), nuclease protection, in situ or microarray hybridization, Western blotting, immunoassays (e.g., ELISA), immunostaining, or fluorescence cell staining.

Also provided are pharmaceutical compositions to reduce the transcriptional activity of SRF and/or MYOCD, as well as processes for using and making these products. The composition is pyrogen-free and further contains a physiologically-acceptable vehicle. It should be noted, however, that a claim directed to a product is not necessarily limited to these processes unless the particular steps of the process are recited in the product claim. SRF- and/or MYOCD-regulated gene expression may be reduced by antisense inhibition, RNA interference, genetic mutation of noncoding (e.g., transcriptional or translational regulatory region) or coding sequences, trans-dominant interference (e.g., a carboxy-terminal deletion of Myocd¹⁷ or splice variant of SRF^{33,34}), or small molecular weight (e.g., less than 3000 MW) soluble inhibitors of gene expression. Alternatively, such agents may be used to decrease FOXO and/or MEF2 expression because they positively regulate MYOCD. Gene transfer of MSX1 and/or MSX2 may be used to increase their expression (MSX1 or MSX2 forms a ternary complex with SRF and MYOCD to inhibit their binding to a CArG box) to inhibit transcriptional activation. A reduction in gene expression may be determined at the level of transcription of DNA to produce RNA, translation of RNA to produce protein, protein activity, or any combination thereof. Screening for chemical inhibitors may be performed by assaying for inhibition of noncoding or coding SRF and/or MYOCD sequences fused to a nuclear localization signal, a protein dimerization domain, a reporter (e.g., alkaline phosphatase, β-galactosidase, chloramphenicol acetyltransferase, β-glucuronidase, luciferases, green or red fluorescent proteins, horseradish peroxidase, β-lactamase, and derivatives thereof), or any combination thereof. Many, but not all, reporters will use a cognate substrate to generate a detectable signal. Inhibition will cause a decrease in the signal detected (e.g., chromogen or fluorescence). Mutations will occur in the SRF and/or MYOCD sequence; chemical inhibitors (e.g., antisense oligonucleotides, siRNA or precursors thereof, dominant negative mutant proteins, natural products, combinatorial synthesis) may be selected from a library of candidate compounds in a cell-free transcriptional assay or a cell-based assay (see Koehler et al., J. Am. Chem. Soc. 125, 8420-8421, 2003; Bailey et al., Proc. Natl. Acad. Sci. USA 101, 16144-16148, 2004). An inhibitor which is selective for SRF- and MYOCD-regulated expression of smooth muscle cell (SMC) contractile proteins is preferred. Nucleic acid inhibitors may be produced by automated synthesis or an expression construct. Protein inhibitors may be produced from an expression construct introduced into a cell by viral infection or transfection. Expression constructs preferably transcribe inhibitors from a regulatory region (e.g., promoter, enhancer) which is vascular cell-specific or derived from a virus, or a combination thereof. The expression construct may be associated with proteins and other nucleic acids in a carrier (e.g., packaged in a viral particle derived from an adenovirus, adeno-associated virus, cytomegalovirus, herpes simplex virus, or retrovirus, encapsulated in a liposome, or complexed with polymers). In vivo treatment includes instillation of a pharmaceutical composition (e.g., virus- or nucleic acid-containing solution) directly into vasculature of the subject. For ex vivo treatment, cells from a subject or donor (e.g., vascular cells or a progenitor thereof) may be virally infected or transfected in vitro and then transplanted into vasculature of the subject. While cell-free transcription assays may be performed to identify inhibitors, (i) cells with mutations that are introduced by random or site-directed mutagenesis or homologous recombination or (ii) cells transfected with an expression construct containing at least a portion of SRF and/or MYOCD and optionally a transcriptional or translational fusion with a reporter can also be assayed. Cells may be vascular cells (e.g., smooth muscle cells), especially of the brain or artery, and more especially of cerebral artery.

### Materials & Methods

### Participants and Neuropathological Diagnosis

VSMC were isolated from rapid brain autopsies from small cortical pial arteries (area 9/10) from 18 individuals. AD patients and age-matched controls were evaluated clinically and followed to autopsy at the AD Research Centers at the University of Southern California and the University of Rochester Medical Center, N.Y. The CDR scores in AD and control individuals were 3-5 and 0, respectively. AD cases were Braak stage V-VI³¹ and CERAD³² frequent to moderate. Controls were Braak 0 or 0-1 and CERAD negative or sparse. See Table 1 for clinical and neuropathological characteristics. The incidence of vascular risk factors (e.g., hypertension, atherosclerosis, etc.), the gender ratio,

TABLE 1A
Aizheimer's Disease Patients

| Patient | | | PMI | | Vascular Risk | | | | |
|---|---|---|---|---|---|---|---|---|---|
| Number | Age | Gender | (hr) | Cause of Death | Factors | Angiopathy | Braak | CERAD | CDR |
| 20 | 70 | M | 5.0 | Pneumonia | None | + | V-VI | Moderate | 4 |
| 41 | 80 | F | | Cardiac | Hypertension | + | V-VI | Frequent | 4 |
| | | | | Arrest | | | | | |
| 42 | 80 | F | 5.2 | Cardiac | Hypertension | + | III-V | Moderate | 4 |
| | | | | Arrest | Atherosclerosis | | | | |
| | | | | | Myocardial | | | | |
| | | | | | Infarction | | | | |
| 43 | 77 | M | 2.8 | Pneumonia | None | + | V-VI | Frequent | 4 |
| 49 | 78 | M | 5.0 | Cardiac | None | + | V-VI | Frequent | 4 |
| | | | | Arrest | | | | | |
| 54 | 73 | M | 2.5 | Pulmonary | Atherosclerosis | + | V-VI | Frequent | 5 |
| | | | | Embolism | | | | | |
| 122 | 99 | F | 3.5 | Cardiac | Atherosclerosis | + | V-VI | Frequent | 5 |
| | | | | Arrest | | | | | |
| 124 | 78 | F | 3.5 | Bowel | Hypertension | + | V-VI | Frequent | 3 |
| | | | | Obstruction | | | | | |

TABLE 1B
Age-Matched Neurologically Normal Subjects (Controls)

| Patient | | | PMI | | Vascular Risk | | | | |
|---|---|---|---|---|---|---|---|---|---|
| Number | Age | Gender | (hr) | Cause of Death | Factors | Angiopathy | Braak | CERAp | CDR |
| 29 | 96 | F | 6.0 | Cardiac | None | − | 0 | Negative | 0 |
| | | | | Arrest | | | | | |
| 38 | 58 | F | 5.5 | Pulmonary | None | − | 0 | Negative | 0 |
| | | | | Embolism | | | | | |
| 39 | 72 | M | 4.3 | Cardiac | Atherosclerosis | − | 0-I | Sparse | 0 |
| | | | | Arrest | Myocardial | | | | |
| | | | | | Infarcation | | | | |
| 40 | 73 | M | 4.7 | Myeloma | Atherosclerosis | − | I-II | Negative | 0 |
| 75 | 86 | F | 3.5 | Cardiac | None | − | 0 | Negative | 0 |
| | | | | Arrest | | | | | |

PMI, Post-mortem Interval; CERAD, Consortium to Establish Registry for Alzheimer's Disease;
CDR, Clinical Dementia Rating Score;
Angiiopathy indicates the presence of CAA.

age, cause of death and the post-mortem interval were comparable between AD and age-matched controls. VSMC from young controls (average age 31.2 years) were isolated from rapid brain autopsies of neurologically normal young individuals with no vascular risk factors autopsied after motor vehicle accidents at the Monroe Medical Examiner Center, Rochester. The cells were harvested under an approved protocol.

### Human VSMC Culture

Pial arterial VSMC was isolated and characterized as previously described⁵¹. Briefly, pial arterial blood vessels from postmortem human brains were dissected, and then digested with 0.1% dispase and 0.1% collagenase in Dulbecco's modified Eagle's medium (DMEM) containing 15 mM Hepes and antibiotics. The minced vessels were first kept at 4° C. for 2 hours, and then incubated at 37° C. for 1.5 hour followed by trituration. Cells were collected by centrifugation and cultured in DMEM containing 10% fetal bovine serum, 1 mM sodium pyruvate, 0.1 mM non-essential amino acids, 100 units/ml penicillin, and 100 μg/ml streptomycin. The cultured VSMC were shown to robustly express vascular smooth muscle cell α-actin, vascular smooth muscle myosin heavy chain, and SM22α.

### Western Blotting

VSMC are washed in cold phosphate buffer saline and then lysed with “crack” buffer (50 mM Tris-HCl, pH, 6.8, 100 mM DTT, 1 mM sodium orthovanadate, 100 μg/ml PMSF, 2% SDS, 10% glycerol, and 1 μg/ml each of pepstatin A, leupeptin, and aprotinin). The lysate is sheared 10× through a 23 g needle, boiled for 10 min, and then spun at 4° C. for 10 min at 14,000 g. The supernatant is collected, quantitated with a protein assay kit (Pierce), and analyzed on a Coomassie-stained polyacrylamide gel for integrity and relative loading. Typically, a denaturing 10% polyacrylamide gel (BioRad MiniProtean) is loaded with 50-100 μg/lane of protein, and then electrophoresed for 1 to 2 hours at 150 V. The gel is transferred to nitrocellulose and then processed for immunoblotting by established methods. Primary antisera and their dilution include SRF (1:1000, Santa Cruz, sc-335), SM-calponin (1:10,000, hCP, Sigma), smooth muscle myosin heavy chain (SM-MHC, 1:500, Santa Cruz, sc-6956), SM α-actin (1:1000, Sigma A-2547), SM22α (1:2000, gift from Dr. Julian Solway, Univ. of Chicago), MYOCD (1:2000, gift from Univ. of Texas Southwestern Antisera Core), and β-tubulin (1:1000, Pharmingen 556321). Following incubation with appropriate secondary antisera, immunoreactive products are detected with a chemiluminescent kit (Pierce). The relative levels of immunoreactive product are measured with a laser densitometer (Molecular Dynamics), and then calculated by normalization to the level of β-tubulin control antibody.

### Quantitative Polymerase Chain Reaction (PCR)

mRNA was quantified using a TAQMAN™ amplification assay (Applied Biosystems) with fluorescently-labeled oligonucleotide probes⁵².

### VSMC Contractile Competence Assay

VSMC were plated in 24-well plates at 4×10⁴ cells/well in DMEM containing 10% fetal bovine serum, 1 mM sodium pyruvate, 0.1 mM non-essential amino acids, 100 units/ml penicillin, and 100 μg/ml streptomycin until 50% to 60% confluent. For contractile activity measurements, DMEM was replaced with physiological salt (Krebs) solution gassed with O₂ and CO₂ (95% and 5%, respectively). After 5 min incubation in Krebs solution, cells were exposed for 2 min to 75 mM KCl in Krebs solution to induce contraction, followed by incubation in KCl-free Krebs solution. For the time-lapse study, VSMC are kept at 37° C. in an incubation chamber on a stage of an inverted microscope (Nikon TE2000-S), and images are captured at 20× magnification using a digital camera (Spot) driven by SimplePCI software package (Compix). The cell length and area are determined at different time points using Image-Pro Plus software. We typically see the peak in contractile activity within 5 to 10 min of KCl stimulation. To determine if calcium is needed for VSMC contraction, 2.5 mM CaCl₂ was left out of the KCl-free and the KCl-containing Krebs solutions, and the assay was performed as described above. We used the same assay to evaluate the effects of adenoviral-mediated MYOCD gene transfer and SRF gene silencing. The maximal cell shortening (contraction) was determined from 100 cells per culture from 3 to 8 different VSMC cultures per group in triplicates.

### Measurement of [Ca²⁺]ᵢ in Single VSMC

The intracellular calcium level of VSMC upon KCl stimulation was imaged using a calcium-sensitive fluorescent dye, Fura-2 AM (Teflabs), as we described⁵³. In brief, VSMC cultured on coverslips were incubated with 4 μM Fura-2 AM in DMEM for 40 min. The coverslips were transferred to a perfusion chamber fitted to a stage of an inverted Nikon Diaphot 300 microscope and superfused with normal Krebs solution for 15 min prior to the stimulation with 75 mM KCl in Krebs solution. [Ca²⁺]ᵢ was measured by digital image fluorescence microscopy (objective, Fluor 40/1.3; Nikon) using Vision 4.0 software (T.I.L.L. Photonics). The fluorescent images were collected with a charge-coupled device (CCD) camera (T.I.L.L. Photonics). Calibrated data were pooled and plotted as mean±s.e.m. of [Ca²⁺]ᵢ.

### Immunostaining of Cerebral VSMC in Human Tissue

For immunohistochemical analysis on brain tissue from AD patients and age-matched controls, we used paraffin sections (6 μm) of frontal cortex (area 9/10) adjacent to the brain surface and pial vessels. Paraffin was removed from sections by washing with xylene; the tissue sections are then rehydrated in a series of decreasing concentrations of ethanol. Antigen retrieval was performed by treating the tissue sections with Retrievagen B (BD PharMingen). The following primary antibodies were used for immunohistochemical analysis: monoclonal mouse antibody against human SRF (1:500, 0.2 mg/ml, Santa Cruz Biotechnology), goat antibody against human MYOCD (1:1000, 0.2 mg/ml, Santa Cruz Biotechnology and gift from Univ. of Texas Southwestern Antisera Core); monoclonal mouse antibody against human smooth muscle specific actin (1:200, 0.2 mg/ml, Oncogene), mouse antibody against human calponin (1:500, 86 μg/ml, DAKO); polyclonal rabbit antibody against human Aβ (1:500, 0.66 mg/ml, Biosource). Primary antibodies were detected with fluorescein or rhodamine-conjugated secondary antibodies, Image analysis was performed with a Nikon fluorescence microscope equipped with a SPOT digital camera. Microvessel size was determined as follows: capillaries to small arterioles (<20 μm), intermediate arterioles and small arteries (20 μm-40 μm and 40 μm-100 μm, respectively), or larger vessels. (>100 μm). Intensity of signal was measured using Image-ProPlus. At least ten randomly selected fields in each region from ten sections were analyzed.

### SRF Silencing by RNA Interference

Briefly, shuttle vectors (pENTR class vectors, Invitrogen) containing the U6-driven SRF RNAi cassette⁴² or the indicated control, were recombined with pAd/pl-DEST (Invitrogen) using LR clonase (Invitrogen) to create the adenovirus constructs. Following linearization with Pacl (New England Biolabs), each adenovirus construct was transfected separately into HEK-293A cells with LIPOFECTAMINE 2000 (Invitrogen). Viral production was allowed to proceed until cell lysis was judged greater than 95% complete, at which time the supernatant was collected. A crude viral lysate was prepared from this supernatant by three freeze-thaw cycles and tested to confirm function. Subsequently, adenovirus was amplified and then purified using the AdenoMini kit from Virapur, per manufacturer's directions. Viral titers, as measured in infectious units (IFU), were determined using the Adeno-X Rapid Titer kit (BD Clontech) per manufacturer's directions. Large-scale adenoviral preparations were kindly provided through the Univ. of Pittsburgh's National Heart Lung and Blood Institute-funded Vector Core Facility. For Western blot analysis of contractile proteins, 2×10⁵ AD VSMC plated in a 60 mm dish were incubated with Ad.shSRF or Ad.shGFP at a multiplicity of infection (MOI) of 100 in DMEM/2% FBS for 2 hours at room temperature with rocking. After removing the virus, transduced AD VSMC were cultured in DMEM for another 4 days. For in vitro contractility assay, 1×10⁴ AD VSMC plated in a 24-well plate were transduced with Ad.shSRF or Ad.shGFP at an MOI of 100, as above.

### MYOCD Gene Transfer

Adenovirus construction was performed essentially as described⁴². Briefly, CMV-driven human MYOCD (kindly provided by Dr. Michael Parmacek) or the indicated control, were recombined with/pAd/pl-DEST (Invitrogen) using LR clonase (Invitrogen) to create the adenoviral constructs. Prior to recombination, a short sequence encoding the FLAG epitope was inserted in-frame at the N-terminus of MYOCD. Linearization with Pacl (New England Biolabs), transfection of HEK-293A cells, viral production, preparation of a crude viral lysate, amplification and purification of adenovirus were as described above. For Western blot analysis of contractile protein profile, age-matched control VSMC were incubated with Ad.MYOCD or Ad. GFP at an MOI of 100. After removing the virus, transduced control VSMC were cultured for 48 hours. For in vitro contractility assay, 1×10⁴ control VSMC plated in a 24-well plate were transduced with Ad.MYOCD or Ad.GFP at an MOI of 100, as above.

### Mouse Vascular Contractility Assay

The thoracic aorta, free from connective tissues, was isolated and removed from anesthetized (0.5 mg/kg ketamine and 5 mg/kg xylazine i.p.) wild type mice using an approved institutional protocol in accordance with National Institutes of Health guidelines. Three to four mm sections were used to determine contraction and relaxation using a 10 ml Radnoti organ bath system and Grass myograph (Grass-Telefactor Instruments). Tissue was bathe in Krebs solution, gassed continuously with 95% O₂ and 5% CO₂ at pH 7.4 and at 37° C.±0.5° C. The resting tension was maintained at 0.5 g. Cumulative dose-response curves for contraction to phenylephrine and relaxation to acetylcholine following pre-contraction with 0.25 μM phenylephrine were determined in aortic rings transduced with Ad.MYOCD or Ad. GFP.

### Transduction of Mouse Arteries

The thoracic aorta was isolated from 3- to 4-month old C57Bl6J mice anesthetized as above. Transduction with MYOCD gene was performed as described for ex vivo arterial preparations^{54,55}. Briefly, two four mm segments were incubated together in a 96-well plate at 37° C. under 95% O₂ and 5% CO₂ for 2 hours with 50 μl of viral suspension containing 2×10⁸ pfu of Ad.MYOCD or Ad.GFP in human endothelial-SFM (Life Technologies) supplemented with 5× insulin/transferrin/selenium (Sigma) and penicillin/streptomycin. After adding 100 μl of endothelial growth medium (RPMI 1640 containing 10% fetal bovine serum, 10% Nuserum, 30 μg/ml endothelial cell growth supplements (Sigma), 5 U/ml heparin, 1 mM sodium pyruvate, 1% non-essential amino acids, 1% vitamins, 25 mM Hepes, 100 units/ml penicillin, and 100 μg/ml streptomycin), the incubation was continued overnight (20 to 24 hours). Detection of β-galactosidase was performed as described⁵⁶. After staining, arterial segments were embedded in OCT, sectioned on a cryostat at 10 μm and photographed at 4× magnification. GFP expression was visualized with an inverted fluorescent microscope (Nikon TE2000-S) and photographed at 10× magnification. For Western blot analysis, aortic rings were rinsed twice with ice-cold PBS, and then each ring was lysed in 25 μl of 1×SDS sample buffer. Lysate (10 μl per lane) was run on a 6% polyacrylamide gel for the detection of myosin heavy chain with mouse monoclonal anti-human antibody (SM-MHC, 1:2,000, Upstate). β-tubulin was used as an internal control for protein loading.

### Transgenic Mice

Tg2576 APPsw^{+/-} mice²¹ were used at 18- to 22-months of age. Brains were removed from anesthetized (0.5 mg/kg ketamine and 5 mg/kg xylazine i.p.) mice using an approved institutional protocol in accordance with National Institutes of Health guidelines. Immunostaining-analysis for SRF and Aβ was performed on 6 μm thick paraffin sections using polyclonal rabbit antibody against human SRF (1:1000, 0.2 mg/ml, Santa Cruz Biotechnology) and human Aβ-specific monoclonal antibody 66.1 (1:500, obtained from Dr. van Nostrand, SUNY Stonybrook).

### Cellular Clearance of Aβ Deposits

This was performed as reported^{48,50}. Multi-spot glass slides were coated with Cy3-labeled Aβ42 (5 μg/spot) without cells, with 500 control cerebral VSMC, with 500 AD VSMC, or with 500 AD VSMC transduced with Ad.shSRF or Ad.shGFP. Cells were incubated for 72 hours and the residual fluorescence Cy3 intensity determined using an inverted microscope (Nikon TE2000-S). The nuclei were visualized by Hoechst staining. Prior to VSMC incubation with Cy3-Aβ42, the relative levels of LRP in cells were determined as described²³ using 5A6 antibody (1:1000; Calbiochem).

### Statistical Analysis

ANOVA was used to determine statistically significant differences. p<0.05 was considered as statistically significant.

### Examples

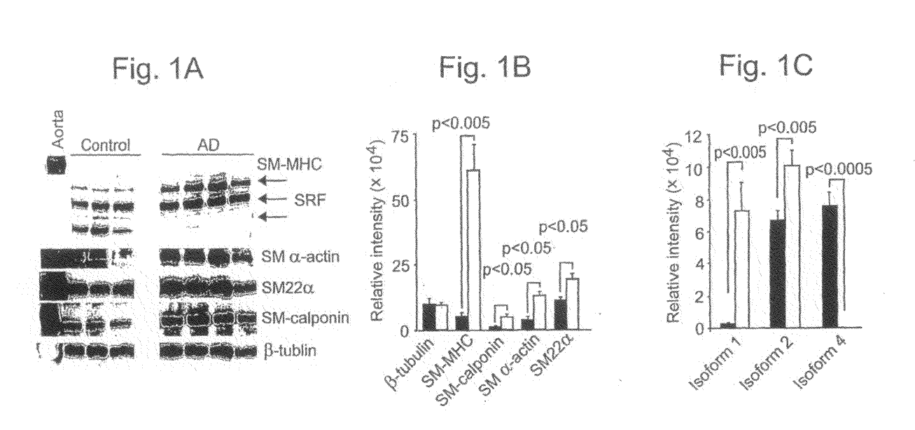

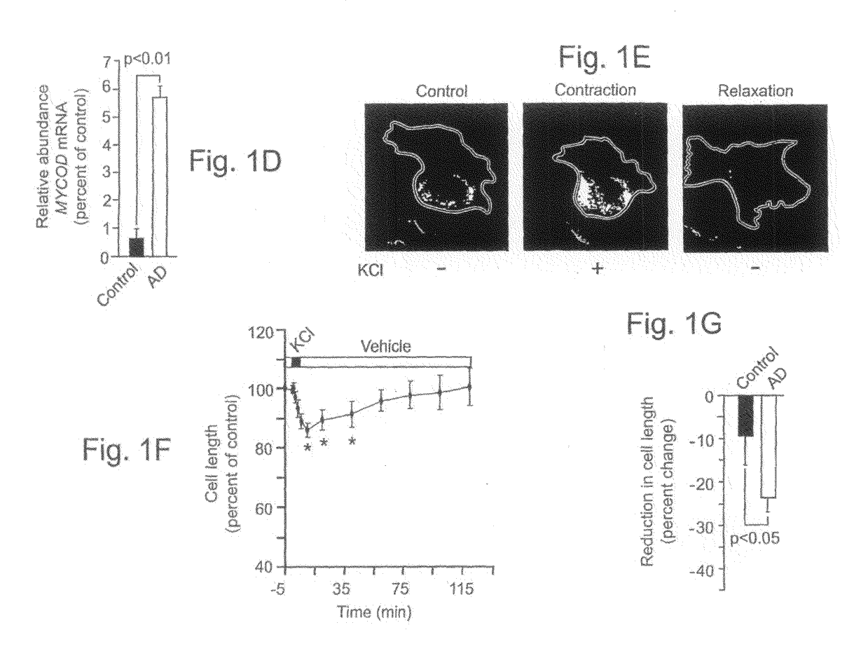

The molecular and cellular basis of Alzheimer arteriopathy has been poorly understood. Here, we analyzed vascular smooth muscle cells (VSMC) derived from small cortical pial and intracerebral arteries which offer the greatest resistance to the blood flow and play a major role in cerebral blood flow (CBF) regulation during brain activation^{1,2}. VSMC were obtained from eight late-stage Alzheimer's disease (AD) patients with severe pathology [Braak—V-VI³¹, CERAD (Consortium to Establish a Registry for Alzheimer's Disease protocol)—frequent or moderate³², clinical dementia rating (CDR) score—4, CAA present, age—79 yrs], five neurologically normal non-demented age-matched controls with no or sparse pathology (Braak—0 or 0-1, CERAD—negative or sparse, dementia score—0, no CAA, age—77 yrs), and five young controls with no pathology (age—32 yrs). There were no differences in gender, cause of death, the postmortem interval (<4 hr) and incidence in the vascular risk factors between AD and age-matched controls (Table 1). First, we noted in a microarray screen that a subset of genes encoding for VSMC-restricted proteins were abundantly represented in AD compared to controls (data not shown). The Western blotting for several such markers¹³ demonstrated that the levels of SMC contractile proteins, i.e., SM myosin heavy chain, SM-calponin, SM α-actin, and SM22α were elevated in AD VSMC compared to age-matched VSMC by 10, 7, 2.5, and 1.7-fold, respectively (FIGS. 1A-1B). There was no significant difference in expression of contractile proteins between age-matched and young controls (not shown). A large number of SMC-restricted genes are regulated by the SRF, a transcription factor that binds a 1.216-fold degenerate cis-element known as a CArG box¹³. The levels of full length SRF were by 23-fold higher in AD VSMC compared to controls (upper arrow in FIG. 1A, isoform 1 in FIG. 1C). In contrast, the lower molecular weight SRF splice variant encoding natural dominant negative isoform of SRF^{33,34} was barely detectable in AD VSMC, but abundantly expressed in control VSMC (lower arrow, FIG. 1A; isoform 4 in FIG. 1C).

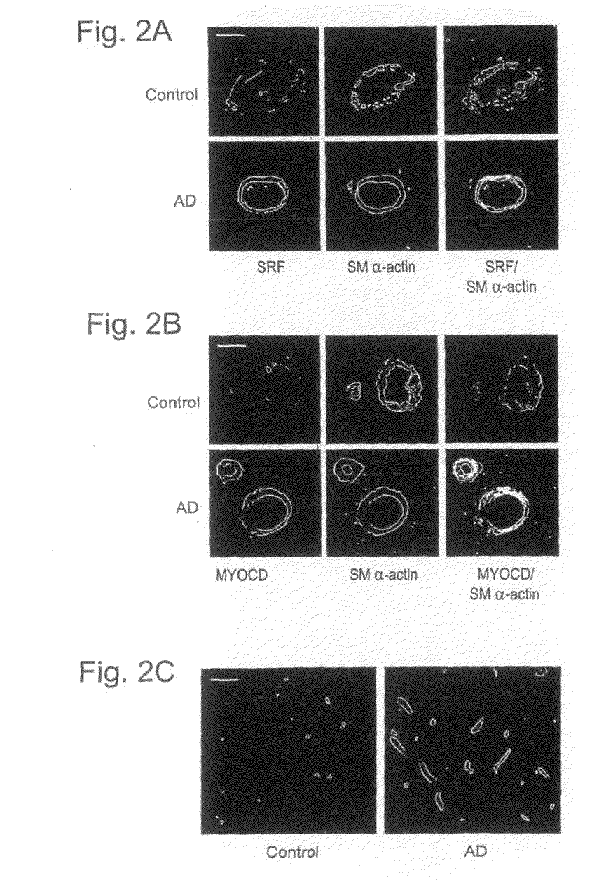

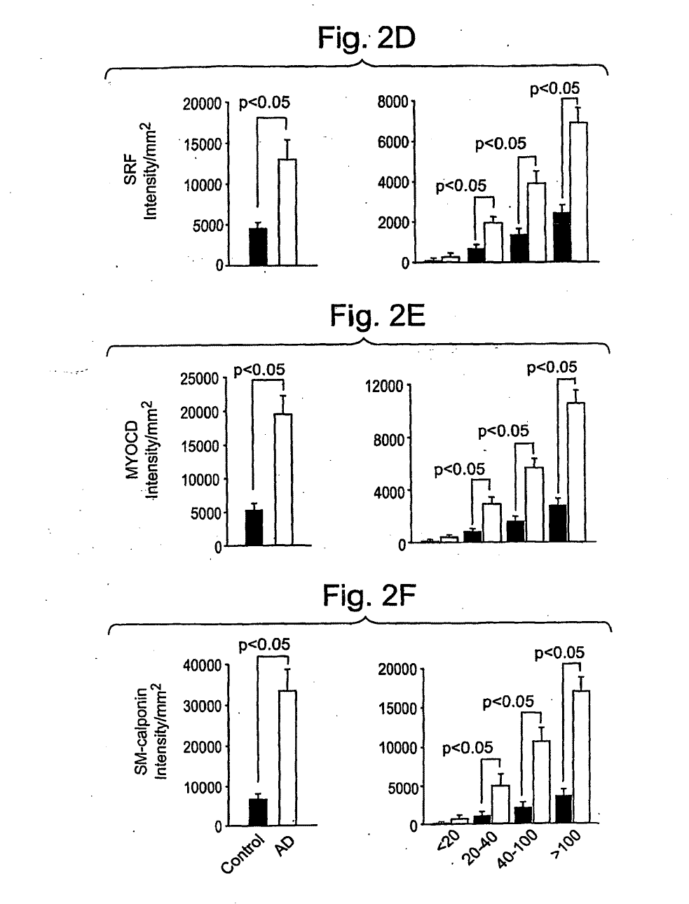

SRF binds a cardiac- and SMC-restricted coactivator MYOCD³⁵. SRF (GenBank Accession numbers NM_{—}003131 and NC_{—}000006 are the mRNA and genomic DNA sequences, respectively) and MYOCD (GenBank Accession numbers NM_{—}153604 and NC_{—}000017 are the mRNA and genomic DNA sequences, respectively) together potently activate a program of SMC differentiation^{13,17,35}. Genetic inactivation of Myocc³⁶ or conditional ablation of Srf³⁷ result in loss of CArG-dependent VSMC gene expression and embryonic death. FIG. 1D shows that AD VSMC express nearly 10-fold higher levels of MYOCD mRNA compared to controls. Double immunostaining analysis of human cortical arterial vessels in brains in situ indicated an overlap between SRF and SM α-actin, and MYOCD and SM α-actin (FIGS. 2A-2B), and substantially increased levels of expression of SRF and MYOCD in AD VSMC compared to control VSMC in arterioles and small arteries of varying caliber from 20-40 μm, 40-100 μm and >100 μm (FIGS. 2D-2E). Consistently, SM-calponin, a known SRF-dependent gene³⁸, was expressed by 6.6-fold higher in AD vessels of different size (FIG. 2C, FIG. 2F) and SM α-actin was increased in AD by 3.5-fold (not shown).

Based on increased expression of contractile proteins in AD VSMC (FIGS. 1A-1D; FIG. 2), we hypothesized that their contractile activity may be higher relative to age-matched control VSMC. FIG. 1C shows VSMC shortening (contraction) in response to potassium chloride (KCl) with a maximal effect at 5 to 10 min after KCl administration (FIG. 1F), and slow return to pre-contraction dimensions (relaxation) (FIGS. 1E-1F). That cell shortening and return to the original pre-KCl dimensions reflect indeed cell contraction and relaxation rather than cellular stress was confirmed by no significant increase in lactate dehydrogenase release, and by phalloidin staining 10 min after KCl exposure indicating the rearrangements of actin stress fibers corresponds to a contractile state (not shown). Cultured SMC are generally refractory to contractile stimulation owing to their phenotypic modulation¹⁴, which may explain relatively slow contraction and relaxation of cerebral VSMC in vitro compared to their rapid responses in vivo².

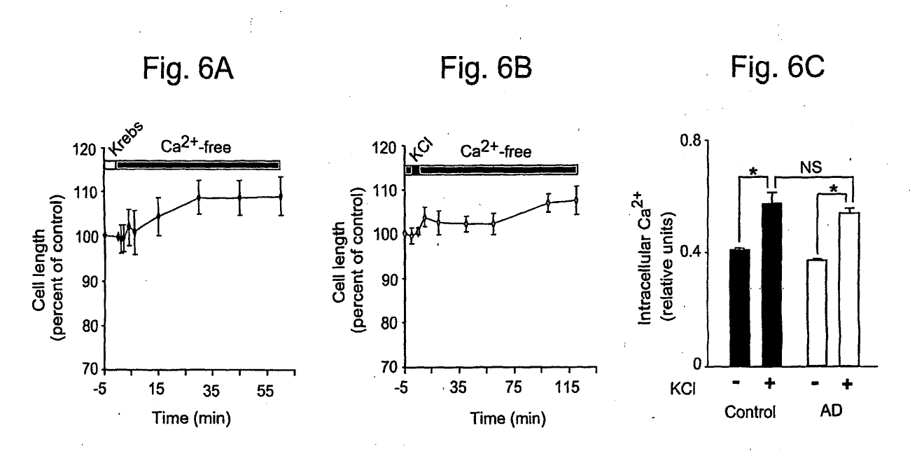

Removal of calcium ions (Ca²⁺) from medium moderately increased the cell length and ablated cell shortening upon KCl administration (FIGS. 6A-6B), confirming extracellular Ca²⁺ is required for VSMC contraction³⁹. An analysis of multiple independent cultures of VSMC (the same ones used in FIGS. 1A-1D; Table 1), demonstrated a statistically significant increase (p<0.05) in KCl-induced cell shortening in AD VSMC compared to control VSMC, i.e., 24.5% vs. 9.2%, respectively (FIG. 1G). To rule out increased Ca²⁺ fluxes as a mechanism for AD VSMC hypercontractility, we measured Ca²⁺ uptake. FIG. 6C shows comparable Ca²⁺ transients between AD and control VSMC consistent with no change in expression of calcium channels as suggested by the microarray data (not shown). Thus, the elevated expression of contractile proteins in AD VSMC correlated well with their inherent ability to hypercontract.

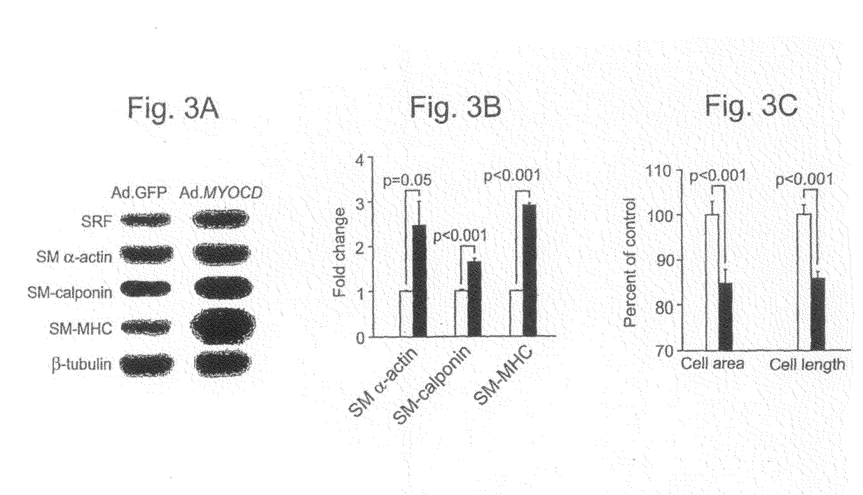

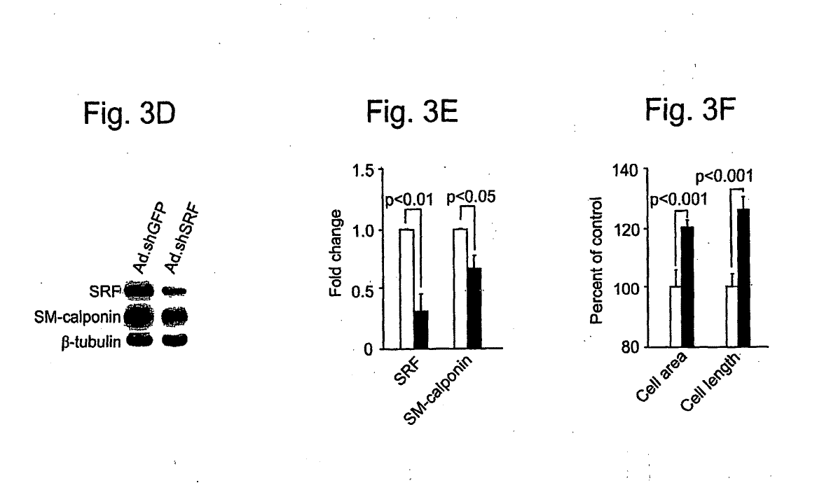

We next hypothesized that overexpressing MYOCD gene in cerebral VSMC would augment contractile protein expression and activity leading to an AD-like phenotype. While MYOCD can elicit a program of SMC differentiation, it has been unclear whether it can promote contractility⁴⁰. Adenoviral-mediated transfer of human MYOCD gene increased dose-dependently MYOCD mRNA expression in VSMC (not shown), and augmented significantly (p<0.01) the levels of contractile proteins, i.e., SM myosin heavy chain, SM-calponin and SM α-actin (FIGS. 3A-3B) consistent with earlier reports^{17,18}. Moreover, MYOCD transfer resulted in increased VSMC contractility (FIG. 3C) compared to GFP-transduced controls. Although MYOCD does not activate the entire SMC gene program⁴¹, our data suggest that MYOCD in human cerebral VSMC can nevertheless direct a functional contractile state which resembles an AD-like hypercontractile VSMC phenotype.

In contrast to MYOCD, silencing SRF in AD VSMC with adenoviral-mediated transfer of short hairpin SRF RNA (Ad.shSRF) reduced expression of SRF by about 70% as well as expression of SRF-dependent VSMC contractile protein SM-calponin (FIGS. 3D-3E). This finding is consistent with our observation that Ad.shSRF effectively reduces endogenous SRF levels and expression of SRF target genes in various cell lines⁴². Silencing of the SRF gene also reduced hypercontractility of AD VSMC (FIG. 3F) suggesting that SRF may be implicated in the development of a hypercontractile VSMC phenotype in AD, probably through its directed expression of VSMC contractile genes.

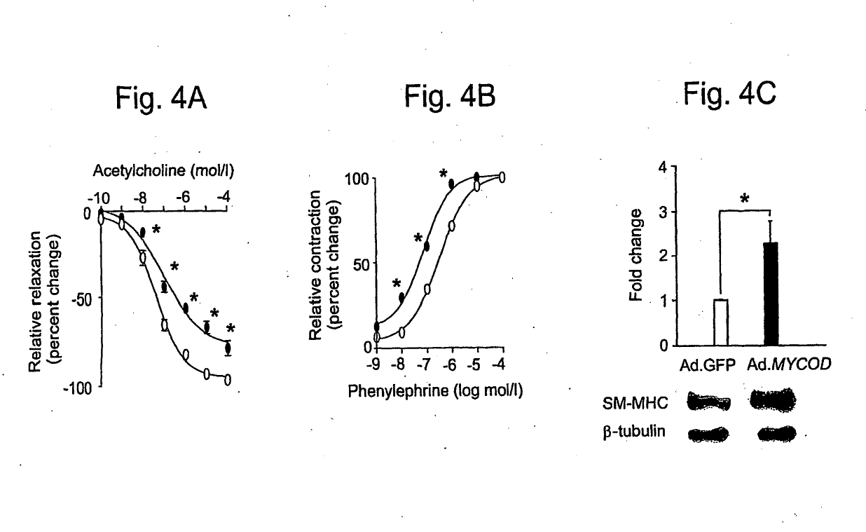

To determine whether the AD-like hypercontractile phenotype can be induced in arteries in a murine model, we transduced ex vivo mouse aortic rings with MYOCD gene or GFP and studied the responses of transduced vessels (inset in FIG. 4C) to acetylcholine, an endothelial-dependent vasodilator which increases nitric oxide production, and to phenylephrine, a direct VSMC vasoconstrictor⁴³. FIGS. 4A-4B show shifts to the right and left of the respective acetylcholine-induced arterial relaxation curve and phenylephrine-induced contraction curve in MYOCD-transduced vessels compared to GFP-transduced controls, suggesting that MYOCD gene transfer reduces arterial vasodilation and amplifies arterial contractility. Consistent with these findings, we also found a 2.2-fold increase in SM myosin heavy chain levels in MYOCD-transduced vessels (FIG. 4C).

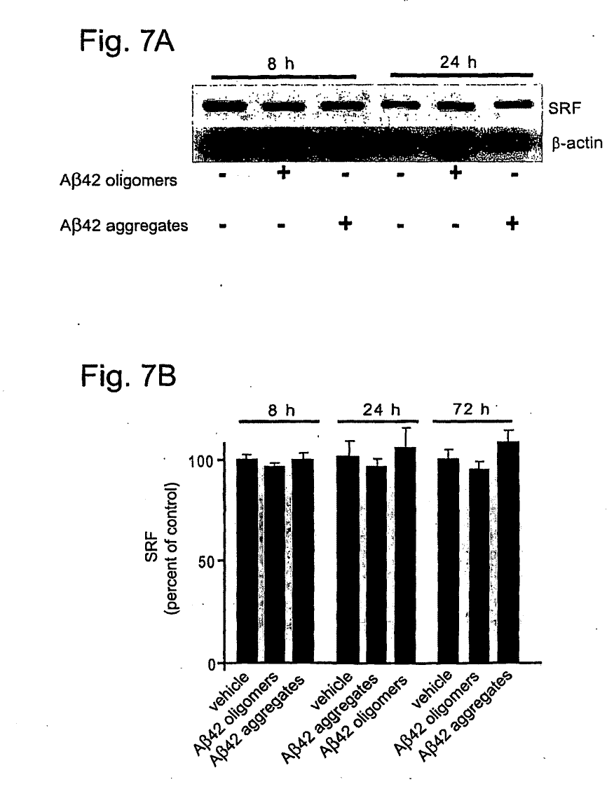

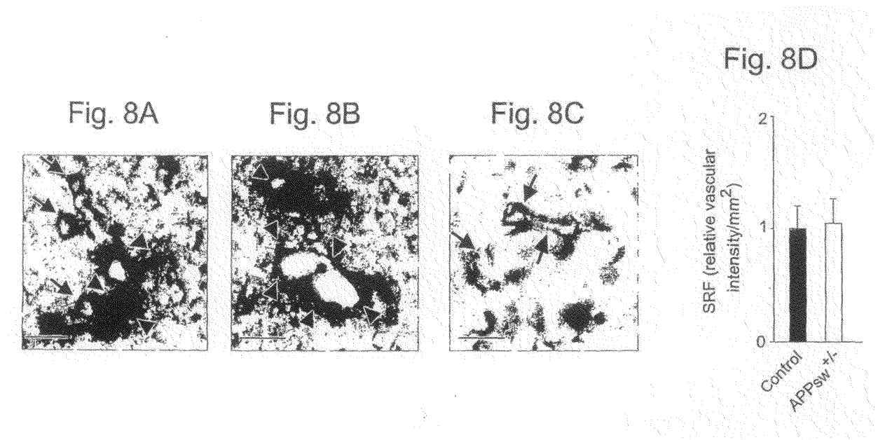

To test whether a link exists between Aβ vascular deposition and SRF expression, we studied the effects of Aβ on SRF expression in human cultured cerebral VSMC. Exogenous pathogenic Aβ42 at different concentrations, structural forms (e.g., oligomers, aggregates)⁴⁴ and over incubation times from 24 to 72 hours did not affect SRF expression (FIG. 7). Next, we studied SRF expression in APPsw^{+/-} mouse model of AD²¹ which develops substantial Aβ brain accumulations after 12 months of age⁴⁵. The SRF-positive vascular profiles and the relative intensity of the vascular SRF signal did not differ significantly between APPsw^{+/-} and age-matched littermate controls at 18-22 months of age (FIG. 8), suggesting exposure to Aβ does not affect the SRF VSMC expression in APPsw^{+/-} mice. Double immunostaining analysis confirmed that SRF-positive vessels were only occasionally positive for Aβ in APPsw^{+/-} mice, whereas most Aβ-positive vessels in APPsw^{+/-} mice were typically negative for SRF (FIG. 8). It has been reported that sublethal concentrations of Aβ42 may lower SRF activity in cultured neurons⁴⁶, but the pathophysiological significance of this finding is unclear. The SRF function in neurons is likely to be different from that in VSMC^{37,47}, and the difference between an earlier study in neurons⁴⁶ and the present findings can be explained by different cell types, as for example, neurons do not express MYOCD.

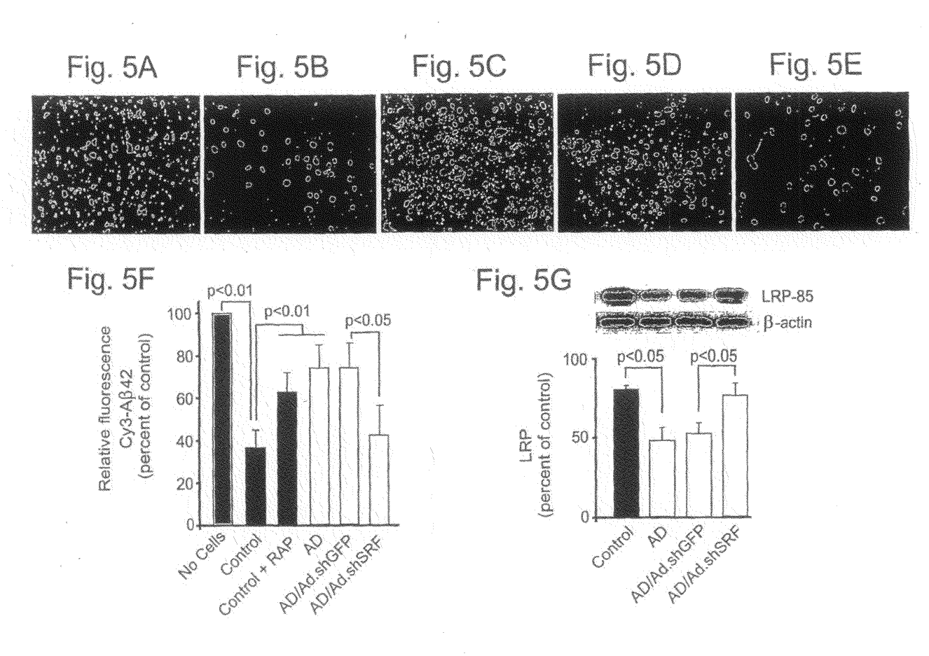

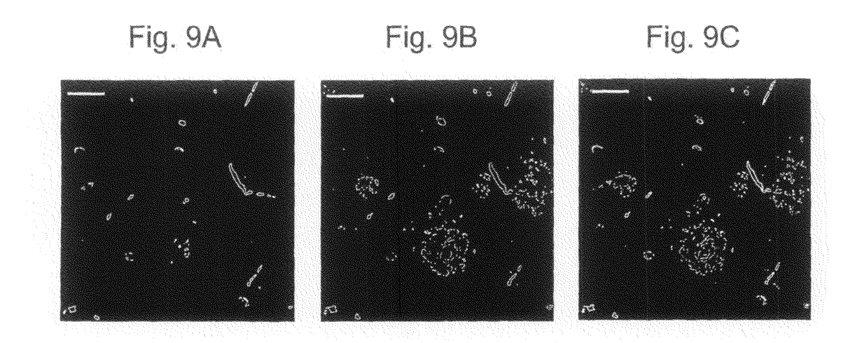

In contrast to data in APPsw^{+/-} mice, we found that a significant increase in SRF-positive vascular profiles in AD (FIGS. 2A and 2D) was accompanied with an increased Aβ vascular immunostaining, and most SRF-positive vessels in AD were positive for Aβ (FIG. 9). This result raised a possibility that although Aβ did not influence the SRF expression in VSMC, an increased SRF activity in VSMC might increase Aβ vascular accumulation. To test this hypothesis, we studied clearance of Cy3-labeled Aβ42 aggregates by AD VSMC using a model similar to that reported in astrocytes⁴⁸, and asked whether silencing the SRF gene influences VSMC-mediated Aβ clearance. We showed that AD VSMC exhibit >70% decrease in Aβ clearance compared to control VSMC (FIGS. 5A-5C, 5F), and that normal cerebral VSMC clear Aβ via the low density lipoprotein receptor related protein 1 (LRP) as demonstrated by significant inhibition with the receptor associated protein, an LRP ligand^{22,23} (FIG. 5B) and anti-LRP antibody (not shown). Demonstration of LRP-mediated Aβ clearance by cerebral VSMC was consistent with previous reports in VSMC⁴⁹, astrocytes⁵⁰, brain endothelial cells and across the blood-brain barrier^{22,23}, whereas reduced clearance of Aβ by AD VSMC was consistent with a significant reduction (p<0.05) in LRP expression (FIG. 5G), as reported for other non-VSMC types of vascular cells in AD^{22,23}. Transduction of AD VSMC with Ad.shSRF, however, improved significantly (p<0.05) Aβ clearance (FIGS. 5D-5E) and increased the levels of Aβ LRP clearance receptor in AD VSMC compared to cells transduced with Ad.GFP (FIG. 5G).

### REFERENCES

1. Zlokovic (2005) Neurovascular mechanisms of Alzheimer's neurodegeneration. Trends. Neurosci. 28, 202-208
2. Iadecola (2004) Neurovascular regulation in the normal brain and in Alzheimer's disease. Nat. Neurosci. Rev. 5, 347-360
3. Greenberg et al. (2004) Amyloid angiopathy-related vascular cognitive impairment. Stroke 35, 2616-2619
4. Vinters & Farag (2003) Amyloidosis of cerebral arteries. Adv. Neurol. 92, 105-112
5. Hossmann (1994) Viability thresholds and the penumbra of focal ischemia. Ann. Neurol. 36, 557-565
6. Mies et al. (1991) Ischemic thresholds of cerebral protein synthesis and energy state following middle cerebral artery occlusion in rat. J. Cereb. Blood Flow Metab. 11, 753-761
7. Martin et al (2000) Local protein synthesis and its role in synapse-specific plasticity. Curr. Opin. Neurobiol. 10, 587-592
8. Debiec et al. (2002) Cellular and systems reconsolidation in the hippocampus. Neuron 36, 527-538
9. Kleim et al. (2003) Functional organization of adult motor cortex is dependent upon continued protein synthesis. Neuron 40, 167-176
10. Snowdon et al. (1997) Brain infarction and the clinical expression of Alzheimer's disease. The Nun study. JAMA 277, 813-817
11. Vermeer et al. (2003) Silent brain infarcts and the risk of dementia and cognitive decline. N. Engl. J. Med. 348, 1215-1222
12. Barber et al. (1999) White matter lesions on magnetic resonance imaging in dementia with Lewy bodies, Alzheimers disease, vascular dementia, and normal aging. J. Neurol. Neurosurg. Psych. 67, 66-72
13. Miano (2003) Serum response factor: Toggling between disparate programs of gene expression. J. Mol. Cell. Cardiol. 35, 577-593
14. Owens et al. (2004) Molecular regulation of vascular smooth muscle cell differentiation in development and disease. Physiol. Rev. 84, 767-801
15. Chen et al. (2002) Myocardin: A component of a molecular switch for smooth muscle differentiation. J. Mol. Cell. Cardiol. 34, 1345-1356
16. Du et al. (2003) Myocardin is a critical serum response factor cofactor in the transcriptional program regulating smooth muscle cell differentiation. Mol. Cell. Biol. 23, 2425-2437
17. Yoshida et al. (2003) Myocardin is a key regulator of CArG-dependent transcription of multiple smooth muscle marker genes. Circ. Res. 92, 856-864
18. Wang et al. (2003) Myocardin is a master regulator of smooth muscle gene expression. Proc. Natl. Acad. Sci. USA 100, 7129-7134
19. Hardy & Selkoe (2002) The amyloid hypothesis of Alzheimer's disease:
Progress and problems on the road to therapeutics. Science 297, 353-356
20. Tanzi et al. (2004) Clearance of Alzheimer's Aβ peptide: The many roads to perdition. Neuron 43, 605-608
21. Hsiao et al. (1996) Correlative memory deficits, Aβ, and amyloid plaques in transgenic mice. Science 274, 99-102
22. Shibata et al. (2000) Clearance of Alzheimer's amyloid-Aβ₁₋₄₀ peptide from brain by low-density lipoprotein receptor-related protein-1 at the blood-brain barrier. J. Clin. Invest. 106, 1489-1499
23. Deane et al. (2004) LRP/amyloid β-peptide interaction mediates differential brain efflux of Aβ isoforms. Neuron 43, 333-344
24. Redwine et al. (2003) The dentate gyrus volume is reduced before onset of plaque formation in PDAPP mice: A magnetic resonance microscopy and stereologic analysis. Proc. Natl. Acad. Sci. USA 100, 1381-1386
25. Iadecola et al. (1999) SOD1 rescues cerebral endothelial dysfunction in mice overexpressing amyloid precursor protein. Nat. Neurosci. 2, 157-161
26. Niwa et al. (2000) Aβ₁₋₄₀-related reduction in functional hyperemia in mouse neocortex during somatosensory activation. Proc. Natl. Acad. Sci. USA 97, 9735-9740
27. Johnson & Albert (2000) Perfusion abnormalities in prodromal Alzheimer's disease. Neurobiol. Aging 21, 289-292
28. Smith et al. (1999) Altered brain activation in cognitively intact individuals at high risk for Alzheimers disease. Neurology 53, 1391-1396
29. Bookheimer et al., (2000) Patterns of brain activation in people at risk for Alzheimer's disease. N. Engl. J. Med. 343, 450-456
30. Ruitenberg et al. (2005) Cerebral hypoperfusion and clinical onset of dementia: The Rotterdam study. Ann. Neurol. 57, 789-794
31. Braak & Braak (1991) Neuropathological staging of Alzheimer-related changes. Acta Neuropathol. 82, 239-259
32. Hyman & Trojanowski (1997) Consensus recommendations for the postmortem diagnosis of Alzheimers disease from the National Institute on Aging and the Regan Institute Working Group on diagnostic criteria for the neuropathological assessment of Alzheimers disease. J. Neuropathol. Exp. Neurol. 56, 1095-1097
33. Kemp & Metcalfe (2000) Four isoforms of serum response factor that increase or inhibit smooth-muscle-specific promoter activity. Biochem. J. 345, 445-451
34. Belaguli et al. (1999) Dominant negative murine serum response factor: Alternative splicing within the activation domain inhibits transactivation of serum response factor binding targets. Mol. Cell. Biol. 19, 4582-4591
35. Wang et al. (2001) Activation of cardiac gene expression by myocardin, a transcriptional cofactor for serum response factor. Cell 105, 851-862
36. Li et al. (2003) The serum response factor coactivator myocardin is required for vascular smooth muscle development. Proc. Natl. Acad. Sci. USA 100, 9366-9370
37. Miano et al. (2004) Restricted inactivation of serum response factor to the cardiovascular system. Proc. Natl. Acad. Sci. USA 101, 17132-17137
38. Miano et al. (2000) Serum response factor-dependent regulation of the smooth muscle calponin gene. J. Biol. Chem. 275, 9814-9822
39. Somlyo & Somlyo (1994) Signal transduction and regulation in smooth muscle cells. Nature 372, 231-236
40. Miano (2004) Channeling to myocardin. Circ. Res. 95, 340-342
41. Yoshida et al. (2004) Forced expression of myocardin is not sufficient for induction of smooth muscle differentiation in multipotential cells. Arterioscler. Thromb. Vasc. Biol. 24, 1596-1601
42. Streb & Miano (2005) AKAP12α: An atypical serum response factor-dependent target gene. J. Biol. Chem. 280, 4125-4134
43. Bai et al. (2004) Pharmacology of mouse isolated cerebral artery. Vasc. Pharmacol. 41, 97-106
44. Kayed et al. (2003) Common structure of soluble amyloid oligomers implies common mechanism of pathogenesis. Science 300, 486-489
45. Kawarabayashi et al. (2001) Age-dependent changes in brain, CSF, and plasma amyloid Aβ protein in the Tg2576 transgenic mouse model of Alzheimer's disease. J. Neurosci. 21, 372-381
46. Tong et al. Beta-amyloid peptide at sublethal concentrations downregulates brain-derived neurotrophic factor functions in cultured cortical neurons. J. Neurosci. 24, 6799-6809 (2004)
47. Ramanan et al. (2005) SRF mediates activity-induced gene expression and synaptic plasticity but not neuronal viability. Nat. Neurosci. 8, 759-767
48. Wyss-Coray et al. (2003) Adult mouse astrocytes degrade amyloid-β in vitro and in situ. Nat. Med. 9, 453-457
49. Urmoneit et al. (1997) Cerebrovascular smooth muscle cells internalize Alzheimer amyloid beta protein via a lipoprotein pathway: implications for cerebral amyloid angiopathy. Lab. Invest 77, 157-166
50. Koistinaho et al. (2004) Apolipoprotein E promotes astrocyte colocalization and degradation of deposited amyloid-β, peptides. Nat. Med. 10, 719-726
51. Davis et al. (2003) Amyloid beta-protein stimulates the expression of urokinase-type plasminogen activator (uPA) and its receptor (uPAR) in human cerebrovascular smooth muscle cells. J Biol. Chem. 278, 19054-19061
52. Holland et al. (1991) Detection of specific polymerase chain reaction product by utilizing the 5′, 3′ exonuclease activity of Thermus aquaticus DNA polymerase. Proc. Natl. Acad. Sci. USA 88, 7276-7280
53. Domotor et al. (2003) Activated protein C alters cytosolic Ca²⁺ flux in human brain endothelium via binding to endothelial protein C receptor and activation of protease activated receptor-1. Blood 101, 4797-4801
54. Ooboshi et al. (1998) Improvement of relaxation in an atherosclerotic artery by gene transfer of endothelial nitric oxide synthase. Arterioscler. Thromb. Vasc. Biol. 18, 1752-1758
55. Jiang et al. (2004) Smooth muscle-specific expression of CYP4A1 induces endothelial sprouting in renal arterial microvessels. Circ. Res. 94, 167-174
56. Lund et al. (2000) Gene transfer of endothelial nitric oxide synthase improves relaxation of carotid arteries from diabetic rabbits. Circulation 101, 1027-1033

Patents, patent applications, books and other publications cited herein are incorporated by reference in their entirety.

All modifications and substitutions that come within the meaning of the claims and the range of their legal equivalents are to be embraced within their scope. A claim which recites “comprising” allows the inclusion of other elements to be within the scope of the claim; the invention is also described by such claims reciting “consisting essentially of” (i.e., allowing the inclusion of other elements to be within the scope of the claim if they do not materially affect operation of the invention) or “consisting of” (i.e., allowing only the elements listed in the claim other than impurities or inconsequential activities which are ordinarily associated with the invention) instead of the “comprising” term. Any of these three transitions can be used to claim the invention.

It should be understood that an element described in this specification should not be construed as a limitation of the claimed invention unless it is explicitly recited in the claims. Thus, the granted claims are the basis for determining the scope of legal protection instead of a limitation from the specification which is read into the claims. In contradistinction, the prior art is explicitly excluded from the invention to the extent of specific embodiments that would anticipate the claimed invention or destroy novelty.

Moreover, no particular relationship between or among limitations of a claim is intended unless such relationship is explicitly recited in the claim (e.g., the arrangement of components in a product claim or order of steps in a method claim is not a limitation of the claim unless explicitly stated to be so). All possible combinations and permutations of individual elements disclosed herein are considered to be aspects of the invention. Similarly, generalizations of the invention's description are considered to be part of the invention.

From the foregoing, it would be apparent to a person of skill in this art that the invention can be embodied in other specific forms without departing from its spirit or essential characteristics. The described embodiments should be considered only as illustrative, not restrictive, because the scope of the legal protection provided for the invention will be indicated by the appended claims rather than by this specification.

1. A method of treating a hypercontractile phenotype of Alzheimer's disease, said method comprising reducing serum response factor (SRF) and/or myocardin (MYOCD) regulated gene expression in at least a cell of a subject's vasculature.

2. The method of claim 1, wherein antisense inhibition causes the reduction.

3. The method of claim 2, wherein the antisense inhibition is mediated by a pyrogen-free composition comprised of an oligonucleotide or an expression construct which produces the oligonucleotide, and a physiologically-acceptable vehicle.

4. The method of claim 1, wherein RNA interference causes the reduction.

5. The method of claim 4, wherein the RNA interference is mediated by a pyrogen-free composition comprised of an siRNA or an expression construct which produces an siRNA precursor, and a physiologically-acceptable vehicle.

6. The method of claim 1, wherein SRF and/or MYOCD trans-dominant interference causes the reduction.

7. The method of claim 6, wherein the trans-dominant interference is mediated by a pyrogen-free composition comprised of an expression construct which produces the dominant negative SRF and/or MYOCD mutant, and a physiologically-acceptable vehicle.

8. The method of claim 1, wherein expression in the cell of one or more contractile proteins is decreased.

9. The method of claim 1, wherein blood flow is increased.

10. The method of claim 1, wherein treatment is performed in vivo.

11. The method of claim 1, wherein treatment is performed ex vivo and the cell is then transplanted.

12. The method of claim 1, wherein the cell is a smooth muscle cell.

13. A method of diagnosing Alzheimer's disease in a subject, said method comprising:
(a) providing a sample of body fluid or tissue from the subject,
(b) determining SRF or MYOCD expression at the level of transcription, translation, or protein activity and
(c) identifying increased SRF or MYOCD expression as a risk factor for existence or development of Alzheimer's disease.

14. The method of claim 13 further comprising identifying vascular hypercontractility as an additional risk factor.

15. The method of claim 13 further comprising identifying diminished vasodilation or enhanced response to vasoconstrictors as an additional risk factor.

16. The method of claim 13 further comprising identifying amyloid angiopathy or reduced blood flow as an additional risk factor.

17. Use of an inhibitor of SRF and/or MYOCD regulated gene expression in smooth muscle cells for the manufacture of a medicament to treat Alzheimer's disease.
